(19) United States
(12) Patent Application Publication
(10) Pub. No.: US 2009/0082630 A1
(43) Pub. Date: Mar. 26, 2009

(54) Disposable flexible endoscope

(76) Inventor: Matthew Tulley, Holmfirth (GB)

Correspondence Address: JAMES C. WRAY, 1493 CHAIN BRIDGE ROAD, SUITE 300, MCLEAN, VA, 22101, US

(21) Appl. No.: 11/919,018

(22) PCT Filed: Apr. 18, 2006
(86) PCT No.: PCT/GB2006/001406

§ 371 Date: Oct. 22, 2007

(30) Foreign Application Priority Data
Apr. 22, 2005 (GB) 0508137.7

(51) Int. Cl. A61B 1/06; H04N 7/18

(52) U.S. Cl. 600/160; 348/65; 348/E07.085

(57) ABSTRACT

A disposable endoscope system (100) connects to a reusable control and monitor unit 6,10 and comprises a flexible insertion section (1) connected to an operating handle (3) that connects to an umbilical cord (4). The end interface (5) of the umbilical is the final component of the disposable endoscope. The flexible insertion section (2) has a video camera (12), an illumination source (11) and an actively steered section that is controlled from the operating handle. The endoscope provides PC mouse functionality, enabling image and procedure logging and controls and integration with other patient management systems.

### BACKGROUND

Endoscopes have been used since the 1960's to view celiac cavities of a patient's body. They are long, slender instruments having a flexible shaft with an objective lens at the distal end and an image transmission system to transmit the image to one or more viewers. Endoscopes are also equipped with an illumination system either at the distal end, or transmitted to the distal end. Flexible endoscopes have a means of directing the distal tip remotely from an operating handle so that the endoscope can be navigated through the body. They also have one or more channels through which air, water or aspiration can be directed as well as instruments. This allows both diagnosis of the patient and therapy, including tissue sampling, injection, electrosurgery and other clinical activities.

Traditionally, endoscopes have been manufactured as reusable instruments and so have to be decontaminated between patients to avoid cross infections. The geometry of the instruments, particularly the fine lumens and valves, makes this difficult and time consuming. Some of the cleaning has been semi automated using flushing cycles, but most of the initial cleaning is still done manually with brushes and can be subject to variability. Because of this there continue to be concerns from users about the cleaning effectiveness of reusable endoscopes. The risk of cross infection can be higher for some patients, e.g. hepatitis or cancer and particularly for these cases a disposable alternative is attractive.

The viability of disposable endoscopes depends on the ‘per procedure’ cost comparison with reusable endoscopes, and decontamination effectiveness and risks. The unit procedure cost for reusable endoscopes depends on their frequency of use, capital cost and their maintenance and decontamination costs. The cost of the disposable flexible endoscope will be minimised by effective use of existing telecommunications and automotive technologies. Also, because the endoscope is not decontaminated, it does not need to be sealed against liquid ingress at the operating handle. This reduces the complexity and cost of the instrument.

Whether disposable or reusable, all endoscopes comprise the aforementioned handle, and by which the endoscope is manipulated by the surgeon during use. On the handle are generally disposed knobs by which the distal end of the endoscope is deflected from an inline position so that curves or corners in the cavity into which the endoscope has been introduced can be negotiated without impacting unduly on the tissues of the cavity. The knobs may pull or release cables that deflect the end. Further knobs or buttons control aspiration and wash fluids. As mentioned above, ports are also provided in the handle to permit insertion of tools.

A further cost associated with reusable endoscopes is their integration with a control unit that is employed to display, and enable manipulation of, the image transmitted from the distal end. Frequently, the image is optically transmitted from the distal end. Indeed, frequently, illumination of the object is provided by the control unit and is transmitted optically to the distal end. Thus the surgeon needs to operate the control unit to display the image he wants to see, frequently involving changing the field of view and the focus of the image. Such optical transmissions are inevitably performed through optic fibres passing through the endoscope. These necessarily imply reliable connection interfaces with the control unit, adding to the cost of the endoscope system.

A further common need of the surgeon, in performing his/her task, is to view patient records. These might comprise details of the medical history of the patient, as well as images employed by other means than the present endoscopic examination, for example, X-ray images or MRI scans. Such records are now generally stored on computer and can be rendered accessible to the surgeon in the operating theatre via a personal computer linked to a central mainframe database.

Thus the surgeon has at least three instruments to operate and it is an object of the present invention to improve on this position and facilitate the introduction of low-cost, single-use endoscopes.

### SUMMARY OF THE DISCLOSURE

In accordance with the present invention there is provided an endoscope system comprising:
a personal computer;
a control unit; and
an endoscope having an insertion section, a handle connected to the insertion section, and an umbilical connected to the handle and detachably connectible to the control unit; wherein
the handle includes mouse buttons operatively connected to the computer to perform cursor control, and left and right click functions of the computer.

Preferably, the insertion unit includes a camera and a light, data comprising the image being transmitted to and from the computer electrically for manipulation, storage and display by the computer.

Preferably, the light comprises a light source powered electrically from the control unit or computer.

Preferably, the computer is communicably connected to the control unit and includes software to manipulate image data from the camera and to display an image on a screen associated with the computer.

Thus the connection between the umbilical and the control unit is relatively uncomplicated comprising only electrical connections, as well as any aspiration and irrigation connections.

Moreover, the control unit does nothing more than transmit electrical signals to and from the computer, as well as providing connections to any such aspiration and irrigation sources. Thus the disposable section of the system, namely the endoscope itself, is relatively low cost, not including any optic fibres or connection interface therefor.

Most importantly, however, the surgeon can now operate the computer while holding the endoscope. This enables him to manipulate the image electronically, through the mouse controls, as well as mechanically via the handle and distal end maneuvering buttons, and to operate the computer to view patient records, all without letting go of the handle and using only one hand, the other being available to perform surgical functions, operate what controls remain on the control unit, type keystrokes on the computer keyboard or manipulate the distal end of the endoscope using the buttons on the handle.

Preferably, the computer includes software to permit control of the control unit by the computer, whereby such aspiration and irrigation may be controlled by the mouse buttons on the endoscope handle.

Thus the control unit provides the interface between the disposable endoscope and the monitoring and fluid control. The control unit is connectible to a supply of irrigation fluid and aspiration. This allows fluids (e.g. water, air) to be admitted into the endoscope, and controls suction from it.

The endoscope is preferably manufactured mainly from plastic materials to minimise unit cost, together with optical and electronic components to capture and transmit the image and provide illumination. The mechanics are based on well established principles from reusable endoscopes however mass production and automotive techniques are used to control costs.

### BRIEF DESCRIPTION OF THE DRAWINGS

A preferred embodiment of the invention will now be described with reference to the accompanying drawings in which:

FIG. 1 shows an overview of the endoscope system;

FIG. 2 is an end view of the distal end of the endoscope of FIG. 1;

FIG. 3 shows the control handle of the system of FIG. 1; and

FIG. 4 is a cross section of the umbilical cord of the system of FIG. 1.

### DETAILED DESCRIPTION OF THE PREFERRED EMBODIMENT

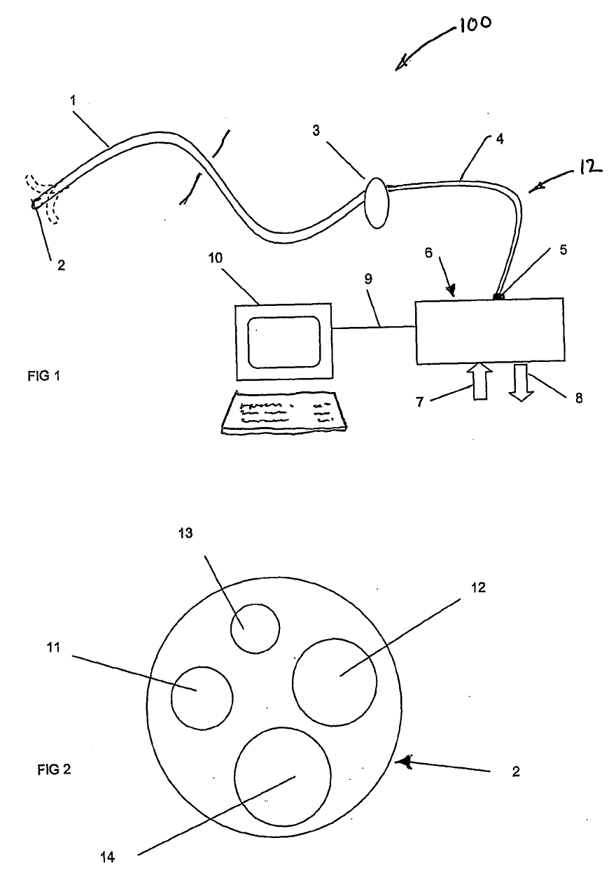

As shown in FIG. 1, an endoscope system 100 comprises a disposable endoscope 12 connected to a reusable control unit 6. The endoscope 12 consists of a flexible insertion section 1 connected to an operating handle 3, which itself connects to an umbilical cord 4. A connection interface 5 at the end of the umbilical 4 is the final component of the disposable endoscope 12.

The connection interface 5 links the endoscope to the control unit 6, which itself is linked to an image display and control personal computer 10 by cable 9.

The control unit 6 is connected to a supply of irrigation fluid 7 and aspiration 8. This allows fluids (e.g. water, air) to be admitted into the endoscope, and controls suction from it.

As shown in FIG. 2, the distal end of the insertion section contains an illumination source 11, a video camera with optics 12, an irrigation channel 13 and a channel 14 for suction or for instruments (not shown). All of the insertion tube is flexible, to allow it to be introduced into non-linear cavities.

The distal section 2 of the insertion tube 2 can be actively steered by controls on the operating handle 3 by means not shown but known to those skilled in the art. Indeed, suitable mechanisms are disclosed in WO2004/086957. The steerable feature of the distal end allows it to be guided through the cavity under inspection.

As shown in FIG. 3 the operating handle 3 is fixed to the proximal end of the insertion section 1 and is used to control and steer it. It is connected to the reusable parts of the system with the umbilical 4. The operating handle is used to steer the distal end of the insertion tube by use of control knobs 15. Access to the channel 14 of the insertion tube 2 is provided by closeable port 16. These enable tools to be inserted through the endoscope. A series of control buttons and switches 17 are provided to control various functions of the endoscope and PC. The functions of these switches can vary and be programmable. However, they conveniently will include a cursor control element, which may take to form of a rollable ball or joystick 17a, “left-click” button 17b, and “right-click” button 17c, each having the same functionality that mouse controls have, as typically employed with a personal computer.

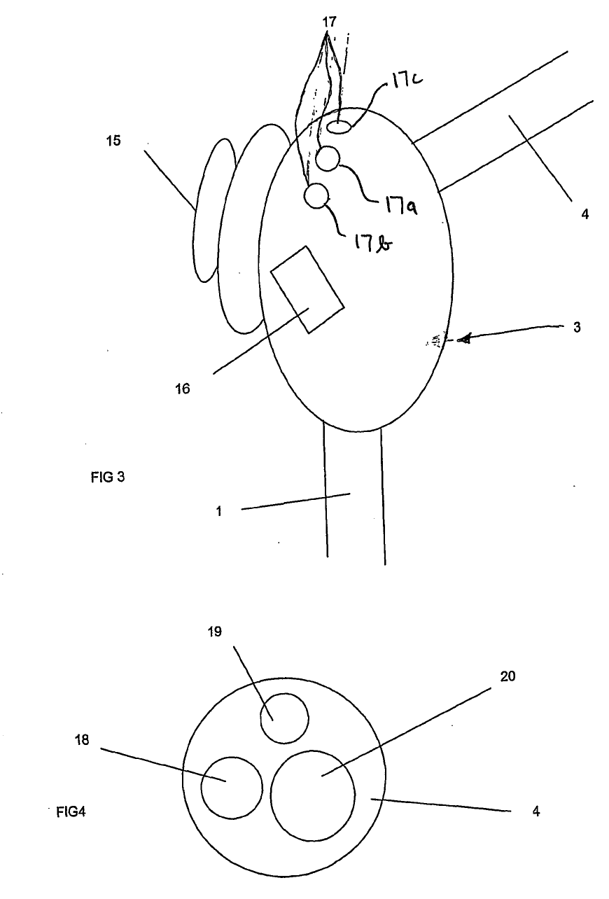

As shown in FIG. 4, the flexible umbilical 4 consists of an irrigation channel 18 that continues through the handle 3 and communicates uninterruptedly with the channel 13 of the insertion end 2. At least, that is the preferred arrangement, with control of the irrigation function (driven by, or under the valve control of the control unit 6) being provided by the computer 10 under the direction of the buttons 17. However, there may equally be a valve control in the handle 3, although simplicity of construction suggests the former approach. Electrical connectors for image transmission and control functions, along with control of the illumination device 11, pass down a channel 19. Again, some connectors pass uninterrupted through the handle 3 into a lumen (not shown) connecting to the light 11 and camera 12, while some terminate at the handle 3. Indeed, some may be connect with switches or other elements such as LEDs in the handle 3, before progressing to the distal end 2.

Finally, an aspiration channel 20 is provided in the umbilical 4 that connects, through the handle 3, with the lumen 14 of the insertion section. This connection is via the port 16. Preferably, as mentioned above, the aspiration is provided by the control unit 6 under computer 10 control, operable from the handle 3 by the mouse buttons 17. However, a manual valve could be incorporated in the handle 3. For example, closing the port 16 could connect suction to the lumen 14, whereas opening the port 16 may be arranged simultaneously to close the channel 20.

While a single umbilical 4 is shown connected to the control unit 6, there is no reason why separate umbilical connections to the computer 10 and to the control unit 6 should not be provided, with a further link (9, as shown) between the computer 10 and control unit 6. In this event, the umbilical connection to the computer is purely electronic, to provide control communication between the mouse controls on the handle 3, as well as image data communication and light source power to the distal end 2 of the endoscope, and the connection between the control unit and handle is purely for aspiration and irrigation ducting. Also, the cable 9, in this event, is purely for control by the computer 10 of valves and/or pumps (not shown) in the control unit 6.

The software needed to provide the computer 10 with the functionality described above is within the normal ability of the person skilled in the art of image data control and manipulation. Likewise, the design of the handle to incorporate computer mouse controls is also within the normal ability of the person skilled in the art. Hence, no further description of these aspects is included herein.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An endoscope system comprising:
a personal computer;
a control unit; and
an endoscope having an insertion section, a handle connected to the insertion section, and an umbilical connected to the handle and detachably connectible to the control unit; wherein
the handle includes mouse buttons operatively connected to the computer to perform cursor control, and left and right click functions of the computer.

2. An endoscope system as claimed in claim 1, in which the connection between the umbilical and the control unit comprises only electrical connections, as well as any aspiration and irrigation connections.

3. An endoscope system as claimed in claim 1 in which the insertion unit includes a camera and a light, data comprising the image being transmitted to and from the computer electrically for manipulation, storage and display by the computer.

4. An endoscope system as claimed in claim 3, in which the light comprises a light source powered electrically from the control unit or computer.

5. An endoscope system as claimed in claim 3, in which said light includes infra-red illumination.

6. An endoscope system as claimed in claim 3, in which the computer is communicably connected to the control unit and includes software to manipulate image data from the camera and to display an image on a screen associated with the computer.

7. An endoscope system as claimed in claim 6, in which said software controls zoom, pan and/or tilt functions of the camera, and is operable by said mouse buttons.
